(19) United States
(12) Patent Application Publication
(10) Pub. No.: US 2021/0330303 A1
(43) Pub. Date: Oct. 28, 2021

(54) Biopsy Device with Focal Internal Grooves and Method of Use

(71) Applicant: H. Lee Moffitt Cancer Center and Research Institute, Inc., Tampa, FL (US)

(72) Inventor: Nam Tran, Thonotosassa, FL (US)

(21) Appl. No.: 17/366,601

(22) Filed: Jul. 2, 2021

Related U.S. Application Data
(63) Continuation of application No. PCT/US20/17859, filed on Feb. 12, 2020.
(60) Provisional application No. 62/804,505, filed on Feb. 12, 2019.

(51) Int. Cl. A61B 10/02

(52) U.S. Cl. CPC A61B 10/0233

(57) ABSTRACT

A medical device and method of using said medical device to remove a biopsy specimen from a patient. The device includes a series of retaining members, such as grooves or ridges protruding into the internal lumen of the medical device. The retaining members do not fully circumscribe the internal lumen. Upon rotation of the device, the retaining members engage the sample tissue received in the internal lumen, which improves the interference between the device and the tissue sample for extraction of the tissue sample from the patient. In some embodiments the grooves are angled to further improve interference between the device and the tissue sample. The medical may also include a biopsy sample ejector or ejector funnel and a cannula and/or a trocar with respective tips configured to optimize penetration of the device into the tissue with minimum patient trauma and enhanced reliability of obtaining a biopsy specimen.

### CROSS-REFERENCE TO RELATED APPLICATIONS

This is a continuation of and claims priority to International Patent Application No. PCT/US20/17859, filed on Feb. 12, 2020, which claims priority to U.S. Provisional Patent Application No. 62/804,505, entitled “BIOPSY DEVICE WITH FOCAL INTERNAL GROOVES AND METHOD OF USE,” filed on Feb. 12, 2019, by the same inventor, the contents of which are herein incorporated by reference.

### BACKGROUND OF THE INVENTION

### 1. Field of the Invention

This invention relates, generally, to a device for tissue biopsy and method of use. More specifically, this invention relates to a tissue biopsy device with internal ridges to retain biopsied tissue.

### 2. Brief Description of the Prior Art

There are many conditions in which access and retrieval of tissue, such as bone marrow and tissue within bone, are important and necessary for diagnostic purposes. These conditions may include, but are not limited to, hematologic diseases, primary bone cancers and bone metastasis from cancers of any origin. In cases of hematologic diseases, there may be a need to treat diseases with bone marrow or stem cell transplants to restore functioning blood cells. Conditions requiring treatment of bone marrow include, but are not limited to, multiple myeloma, lymphoma and leukemia. In primary or metastatic bone cancers, accurate diagnoses are required to treat with chemoradiation. Primary bone cancers may include, aneurysmal bone cysts, osteosarcoma, chondrosarcoma, or Ewing sarcoma, while the most common bone metastatic cancers include, breast, lung, prostate, melanoma, and renal.

Biopsy needles are commonly used in the diagnostic procedures described above. There is, however, often a lack of adherence between the biopsied tissue sample and biopsy needle when performing the biopsy. A lack of adhesion makes it difficult to extract the tissue sample. Current biopsy needles are relatively small, which makes the retrieval process even more difficult. Currently, about 40% of biopsies fail to extract a good tissue sample because the lack of adherence between the biopsy needles and the tissue samples.

Currently available devices and techniques, such as those found in U.S. Pat. No. 9,717,564 to Miller et al. and U.S. Patent Publication No. 2018/0000465 to Brown et al., include intraosseous (JO) needles with a removable trocar disposed on the needle. Various shapes and sizes of handles may be used to apply manual pressure and to manually rotate the IO needle and removable trocar as a set. These manual IO devices often result in a core specimen of bone, but fail to ensure that bone marrow is always successfully retrieved. As a result, multiple insertions at different sites are required to obtain a satisfactory bone and/or bone marrow biopsy specimen. Obviously, multiple insertions increase the patient's discomfort. In addition, it can predispose the patient to spinal fractures or instability. Lastly, inadequate tissue sampling or missed target can result in a missed diagnosis and delayed medical treatment.

Both disclosures identified above disclose a retaining structure having a helical shape. A helical-shaped retaining structure has three major downsides. The first, is that the helical structure requires the biopsy needle to be inserted in a rotational manner. If the biopsy needle is not rotated, the helical shape effectively reduces the diameter of the opening of the biopsy needle and the tissue that passes into the biopsy needle becomes a cylindrical piece with an outside diameter equal to the inner diameter of the helical retaining structure. These biopsy needles have the same effect as a biopsy needle having a distal opening with a diameter equal to the inner diameter of the helical thread if the biopsy needle is not rotated into the tissue. This issue is further exacerbated due to the fact that it can be difficult to tell when the needle has reached an insertion surface of the desired tissue and it can be difficult to determine the amount of pressure required to rotationally penetrated the tissue without cylindrically carving the sample into a smaller diameter. The difficulty in determining an appropriate insertion pressure is especially understandable when considering the fact that biopsy needles can be used to retrieve various types of tissue ranging from bone to soft tissue.

The second downside is that the helical retaining structure occupies more space within the internal lumen of the biopsy needle, which reduces the amount of tissue that can be retrieved. Biopsy needles are particularly small in diameter without a retaining structure. Moreover, most biopsy devices include an introductory needle configured to restrict the axial movement of the biopsy needle in a distal direction for various safety reasons. As a result, there is only a limited amount of space in the internal lumen of the biopsy needle that can recover tissue. For that reason, it is important to reduce the size of the obstructions on the internal lumen of the biopsy needle. The helical-shaped retaining structure significantly and unnecessarily reduces the already limited space available to retrieve tissue samples.

The last major downside to helical-shaped retaining structures is an inability to easily and inexpensively manufacture a biopsy needle with a helical-shaped retaining structure. Biopsy needles typically have a diameter of 1.5875 mm Welding or extruding a helical retaining structure to the inner surface of a biopsy needle is nearly impossible because of the size of the biopsy needle. Biopsy needles that are manufactured with an internal helical-shaped retaining structure are unreasonably expensive to produce resulting in an end product that is far too expensive for the end purchaser.

Accordingly, what is needed is a medical device that both improves the likelihood and ease of retrieving an adequate tissue sample from a patient without significantly reducing the amount of tissue that can be retrieved, increasing the cost to manufacture the device, or increasing the difficulty in using the device. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the instant application, Applicant in no way disclaims these technical aspects, and it is contemplated that the instant application may encompass one or more of the conventional technical aspects discussed herein.

The present disclosure may address one or more of the problems and deficiencies in the art discussed above. However, it is contemplated that this disclosure may prove useful in addressing other problems and deficiencies in many technical areas. Therefore, the present application should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for a medical device that both improves the likelihood and ease of retrieving an adequate tissue sample from a patient without significantly reducing the amount of tissue that can be retrieved, increasing the cost to manufacture the device, or increasing the difficulty in using the device is now met by a new, useful, and nonobvious invention.

The novel structure includes a medical device configured to biopsy a tissue sample from a patient. The medical device includes a biopsy needle having an elongated body with an outer surface and an internal lumen established by an inner surface. The inner surface includes a plurality of discontinuous, longitudinally spaced retaining members extending inwardly towards a central longitudinal axis of the elongated body. Each of the retaining members does not fully extend around a circumference of the inner surface.

The biopsy needle further includes a distal aperture in a distal end of the biopsy needle providing access to the internal lumen. The biopsy needle can be inserted into tissue in the patient, such that a portion of tissue passes through the distal aperture and into the internal lumen. Then the biopsy needle can be rotated to cause the plurality of retaining members to engage and retain the portion of tissue within the internal lumen when the biopsy needle is retracted out of the patient.

In an embodiment, each of the plurality of retaining members is angled with respect to the central longitudinal axis, such that an acute angle between the longitudinal axis and each retaining member is between 45 and 90 degrees.

In an embodiment, the medical device further includes a handle integrated with or attachable to a proximal end of the biopsy needle to enable an operator to rotate the biopsy needle. In an embodiment, the distal end of the biopsy needle includes a beveled cutting surface.

An embodiment includes an introducer needle configured to house the biopsy needle within an internal lumen when the medical device is inserted into the patient. The biopsy needle can distally extend a predetermined distance from a distal end of the introducer needle and the biopsy needle includes the plurality of retaining members longitudinally spaced along a length of the biopsy needle that is equivalent to the predetermined distance that the biopsy needle can distally extend from the distal end of the introducer needle.

The present invention further includes a method to biopsy a tissue sample from a patient. The method includes retrieving a biopsy needle having an elongated body with an outer surface and an internal lumen established by an inner surface; inserting the biopsy needle into tissue within a patient in a non-rotational manner, such that a portion of tissue passes through the distal aperture and into the internal lumen; upon the biopsy needle reaching an operator's preferred depth within the tissue, rotating the biopsy needle such that the plurality of retaining members are rotated into engagement with the tissue within the internal lumen; and retracting the biopsy needle from the patient. In an embodiment, the step of rotating the biopsy needle includes at least a 90-degree rotation about the central longitudinal axis.

The retrieved biopsy needle includes an inner surface having a plurality of discontinuous, longitudinally spaced retaining members extending inwardly towards a central longitudinal axis of the elongated body. Each of the retaining members does not fully extend around a circumference of the inner surface. The biopsy needle further includes a distal aperture in a distal end of the biopsy needle providing access to the internal lumen. The plurality of retaining members retain the portion of tissue within the internal lumen when the biopsy needle is retracted out of the patient.

In an embodiment, a proximal end of the biopsy needle is integrated with or attachable to a graspable handle to enable an operator to rotate the biopsy needle. In an embodiment, the distal end of the biopsy needle includes a beveled cutting surface.

In an embodiment, each of the plurality of retaining members is angled with respect to the central longitudinal axis, such that an acute angle between the longitudinal axis and each retaining member is between 45 and 90 degrees.

An embodiment of the present invention includes a method of manufacturing a biopsy needle. The method includes retrieving a tubular biopsy needle having an elongated body with an outer surface and an internal lumen established by an inner surface and crimping the outer surface of the elongated body at a location proximate to a distal end of the elongate body and at a plurality of longitudinally spaced locations. Each crimping creates a retaining member that extends within the internal lumen towards a central longitudinal axis of the elongated body. In addition, each retaining member does not fully extend around a circumference of the inner surface.

In an embodiment, each crimping occurs at an angle with respect to the central longitudinal axis, such that an acute angle between the longitudinal axis and each retaining member is between 45 and 90 degrees. In an embodiment, each crimping is longitudinally aligned along the outer surface of the elongated body. In an embodiment, each crimping creates a retaining member extending generally the same distance towards the central longitudinal axis of the elongate body.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

While the specification concludes with claims particularly pointing out and distinctly claiming what are regarded as embodiments of the invention, the advantages of embodiments of the disclosure may be more readily ascertained from the description of certain examples of embodiments of the disclosure when read in conjunction with the accompanying drawings, in which:

FIG. 1 depicts a perspective view of a device for bone and/or bone marrow access or biopsy according to an embodiment of the invention.

FIG. 2A is a side perspective view of a prior art device.

FIG. 2B is a side perspective view of an embodiment of the present invention.

FIG. 2C is a side perspective view of an embodiment of the present invention.

FIG. 3A is an end view of a prior art device viewed from the distal end.

FIG. 3B is an end view of an embodiment of the present invention viewed from the distal end.

FIG. 4 depicts a detailed longitudinal axis cross-sectional view of an embodiment of the present invention.

FIG. 5 is a perspective view of an embodiment of the present invention.

FIG. 6 is a perspective view of an embodiment of the present invention providing an upward view into the distal end of the device.

FIG. 7 is a side view of an embodiment of the present invention.

FIG. 8 is an end view of an embodiment of the present invention looking down the distal end of the device.

FIG. 9 is a side view of cross-section of an embodiment of the present invention.

FIG. 10 is a close-up view of detail C from FIG. 9 FIG. 11 is a top view of an embodiment of the present invention.

FIG. 12 is a flowchart of an embodiment of the method of present invention.

FIG. 13 is a flowchart of an embodiment of the method of manufacturing the present invention.

### DETAILED DESCRIPTION OF THE INVENTION

In the following detailed description, reference is made to the accompanying drawings, which form a part thereof, and within which are shown by way of illustration specific embodiments by which the invention may be practiced. It is to be understood that other embodiments may be used, and structural changes may be made without departing from the scope of the present application. These embodiments are described in sufficient detail to enable those of ordinary skill in the art to practice the present disclosure, and it is to be understood that other embodiments may be utilized, and that structural, logical, and electrical changes may be made within the scope of the disclosure.

From the following descriptions, it should be understood that components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

As used herein, the term “subject,” “patient,” or “organism” includes humans and animals (e.g., mice, rats, pigs, cats, dogs, and horses).

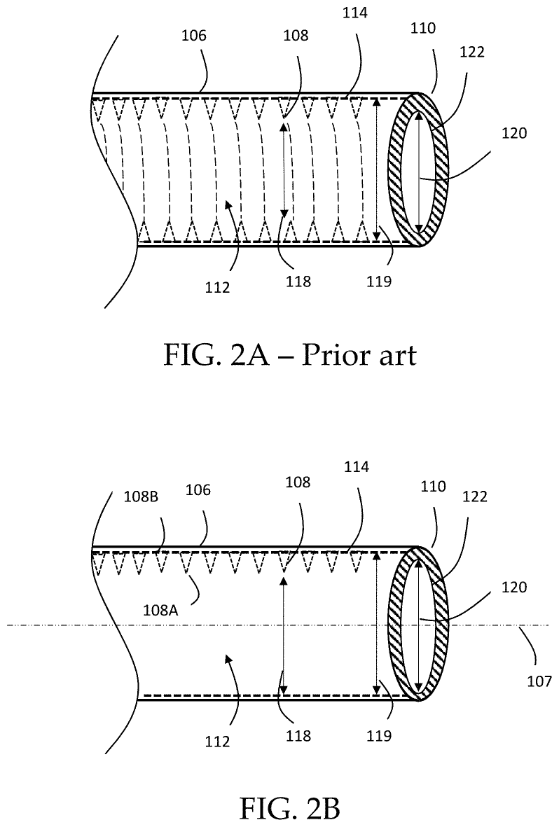

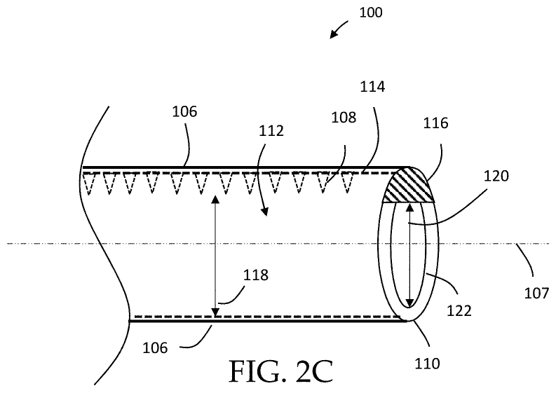

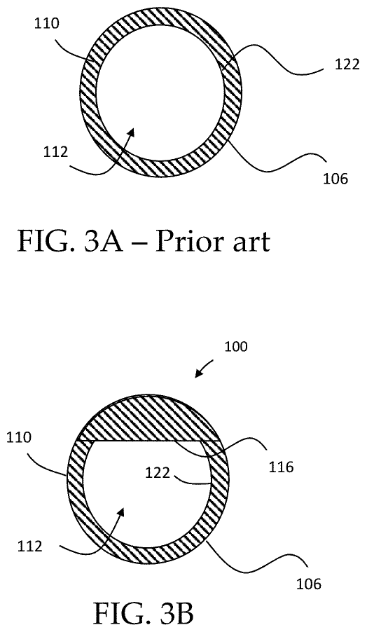

The present invention includes a medical device, such as a biopsy needle, a method of use, and a method of manufacturing the biopsy device. The biopsy device is adapted to better retrieve and retain tissue samples from a patient in comparison to prior art devices such as those depicted in FIGS. 2A and 3A. These prior art devices include helical-shaped retaining members 108 in an attempt to improve tissue retrieval. The use of helical-shaped retaining members, however, has produced poor results.

The helical shape results in a biopsy needle that must be inserted in a rotational manner. If the biopsy needle is not rotated during insertion or if the biopsy needle is inserted with too much axial force, the helical shape cylindrically carves the tissue sample and effectively reduces the diameter of the opening of the biopsy needle to the diameter identified by arrow 118. Rather than retrieving a tissue sample with a diameter generally the size of the internal diameter 119 of internal lumen 112 or even a tissue sample with a diameter generally the size of diameter 120 of distal aperture 122, the tissue sample that passes into the biopsy needle becomes a cylindrical core with an outer diameter equal to the inner diameter 118 of helical-shaped retaining members 108. These biopsy needles have the same effect as a biopsy needle having a distal aperture 122 with a diameter equal to the inner diameter of the helical thread 108 if the biopsy needle is not rotated into the tissue. In this instance, the helical-shaped retaining members 108 are rendered useless because they cannot penetrate the outer surface of the tissue sample.

This issue is further exacerbated due to the fact that the it can be difficult to tell when the needle has reached the desired tissue and it can be difficult to determine the amount of pressure required to rotationally penetrated the tissue without cylindrically carving the sample into a smaller diameter. The difficulty in determining an appropriate insertion pressure is especially understandable when considering the fact that biopsy needles can be used to retrieve various types of tissue ranging from hard bone to soft tissue.

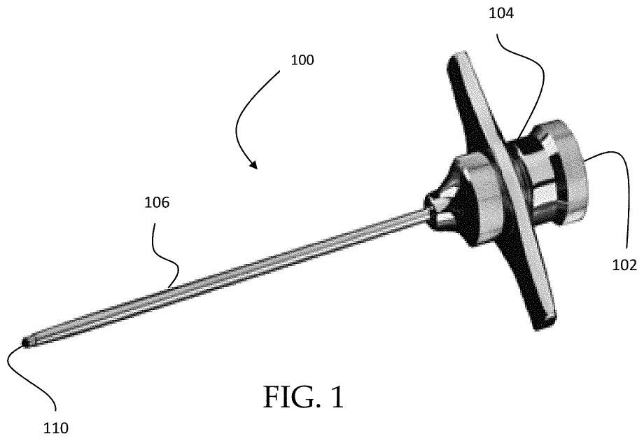

The present invention provides a solution of this issue. As depicted in FIG. 1, an embodiment of the medical device includes biopsy needle 100 having elongated tubular body 106 (also referred to as a shaft) extending between proximal end 102 and distal end 110. The medical device further includes handle 104 disposed proximate to proximal end 102 to allow an operator to rotate the device during use.

As best depicted in FIG. 2B, an embodiment of the present invention includes body 106 having external surface 115 and internal surface 114 that establishes internal lumen 112. Biopsy needle 100 further includes distal aperture 122 axially disposed in distal end 110. Distal aperture 122 is in open fluid communication with internal lumen 112. Biopsy needle 100 can therefore axial penetrate tissue and a sample of the tissue will pass through distal aperture 122 and into internal lumen 112.

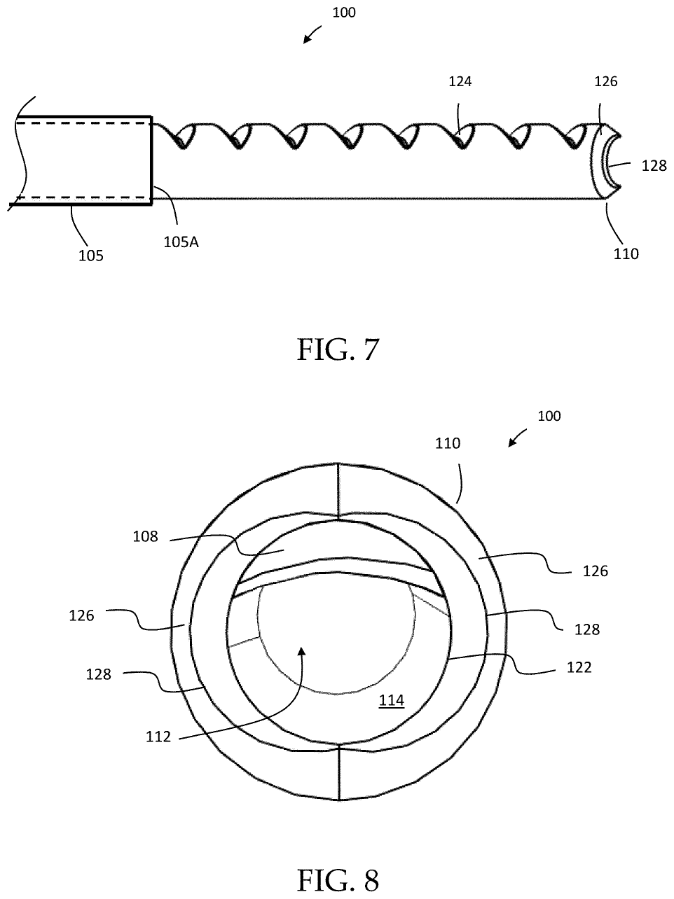

A plurality of longitudinally spaced retaining members 108, depicted as partial threads or grooves 108 in FIG. 2B, extend from internal surface 114 towards central longitudinal axis 107 of internal lumen 112 of biopsy needle 100. The plurality of longitudinally spaced retaining members 108 starts at, or proximate to, distal end 110 of the biopsy needle 100 to ensure that retaining members 108 can engage the sample tissue that passes into internal lumen 112. In an embodiment, the plurality of longitudinally spaced retaining members 108 extend a distance from distal end 110 towards proximal end 102 that is at least the distance that biopsy needle 100 is permitted to distally extend beyond introducer needle 105 (see FIG. 7). In an embodiment, the plurality of longitudinally spaced retaining members 108 extends at least 10 cm from distal end 110 towards proximal end 102, which is a typical distance that a biopsy needle is permitted to distally extend beyond introducer needle 105. In an embodiment, the first retaining member starts within 5 cm from distal end 110 and the plurality of longitudinally spaced retaining members 108 extends at least 10 cm from distal end 110 towards proximal end 102. In an embodiment the plurality of longitudinally spaced retaining members 108 extends the entire distance from distal end 110 to proximal end 102.

As depicted in FIG. 2B, retaining members 108 are non-circumferentially disposed on internal surface 114 of elongated body 106 and extend towards central longitudinal axis 107. Distance 118 between the inward most edges 108A of retaining members 108 and the opposite side of internal surface 114 is greater than if retaining members 108 extended circumferentially around internal surface 114. This fact is illustrated in FIG. 2A which depicts a prior art device having circumferentially extending, helical-shaped retaining members 108. In comparing FIG. 2A to FIG. 2B, it is clear that the distance, identified by arrow 118, between retaining members 108 is less when retaining members 108 circumferentially extend about internal surface 114. Having a larger lumen increases the odds of collecting an adequate tissue sample during operation of the biopsy needle.

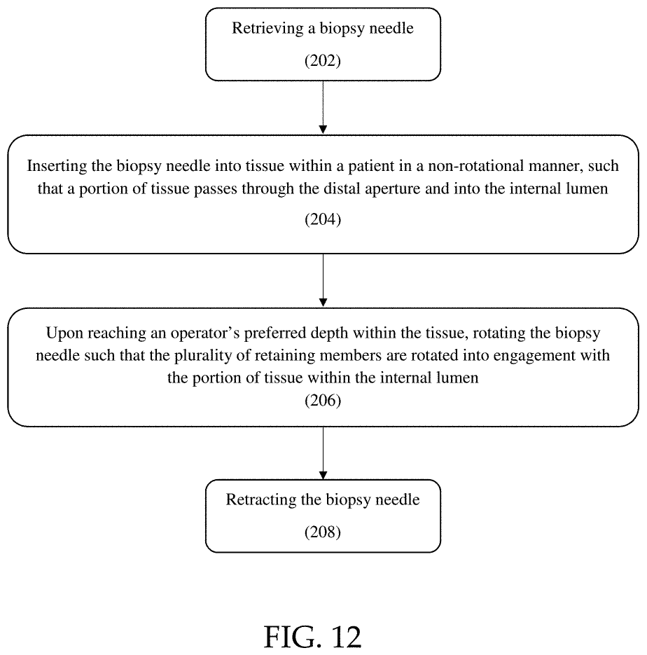

Moreover, non-circumferentially extending retaining members 108 result in a biopsy needle that is easier to operate and is more effective at retaining tissue samples. Biopsy needle 100 of the present invention can axially penetrate tissue without needing to be rotated during penetration. Once at the desired depth, a user can rotate the biopsy needle to bring retaining members 108 into engagement with the tissue sample to retain the tissue sample during extraction. This method of operation will be further discussed below and is depicted in FIG. 12.

In an embodiment, retaining members 108 have an outermost lateral section 108B that is integrated with or attached to internal surface 114 of elongated body 106 and an innermost lateral section 108A that extends towards central longitudinal axis 107. In an embodiment, the cross-sectional shape of each retaining member 108, when viewed from an end view, is generally D-shaped, similar to the retention flange 116 which is depicted in FIG. 3B, or crescent-shaped as depicted in FIG. 8. Various embodiments, may employ retaining members having various other cross-sectional shapes.

In an embodiment, a single retaining member 108 is disposed within internal lumen 112. Retaining member 108 does not fully circle the circumference of the internal surface 114 for the reasons stated above. Retaining member 108 resides at distal end 110 or between distal end 110 of biopsy needle 100 and distal end 105A of introducer needle 105 (see FIG. 7). In an embodiment, the single retaining member 108 resides within 10 cm from distal end 110 of biopsy needle 100. A single non-circumferential retaining member can be used for certain tissue that can be retained by a single retaining member. Alternatively, an embodiment may include two or more retaining members 108 for use with tissue that requires more than one retaining member to remain within the internal lumen when the biopsy needle is retracted from a patient.

Referring now to FIGS. 2C and 3B, an embodiment of the present invention includes a plurality of longitudinally spaced, non-circumferential, discontinuous retaining members 108 and also includes a non-circular distal aperture 122 to aid in retaining tissue samples. As shown in FIG. 3A, conventional biopsy needles include distal aperture 122 established by a circular opening. The circular shape of distal aperture 122 establishes the maximum size of retrieved tissue and fails to include a retaining component, other than helical threads 108, which are inherently flawed as explained above. In contrast, non-circular distal aperture 122 of the present invention as depicted in FIGS. 2C and 3B provides an additional retention flange 116 that retains tissue when biopsy needle 100 is rotated after having been fully inserted into a mass of tissue in a non-rotational manner.

The illustrated embodiment in FIGS. 2C and 3B includes non-circular distal aperture 122 being generally D-shaped or crescent-shaped as established in part by retention flange 116. Retention flange 116 resides at the distal end of shaft 106 and extends towards central longitudinal axis 107 of shaft 106 to reduce the area of distal aperture 122. In doing so, tissue can enter distal aperture 122 and after the tissue sample is received within internal lumen 112, an operator can rotate shaft 106 to bring the tissue sample proximally behind retention flange 116. Once shaft 106 is rotated to bring at least a portion of the tissue behind flange 116, the operator can more confidently remove the shaft and tissue from the patient without fear of the tissue inadvertently exiting shaft 106 through distal aperture 122.

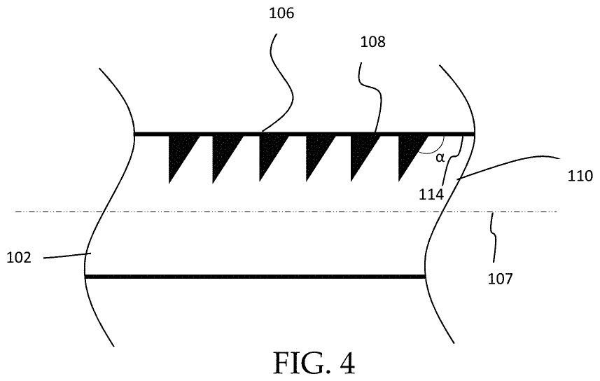

As depicted in FIG. 4, an embodiment of retaining members 108 includes leading edges (distal edges) that are angled from distal end 110 toward proximal end 102 of shaft 106 of the biopsy needle. With respect to the longitudinal extent of internal surface 114, the leading edges are angled in accordance with angle α. Angled retaining members 108 facilitate penetration of the retaining members 108 into tissue while increasing the retention of the tissue sample once it is inside shaft 106 to help ensure that the tissue sample remains within shaft 106 when the biopsy needle is retracted out of the patient's body. In an embodiment, retaining members 108 form an angle α of at least 5° from the longitudinal extent of internal surface 114. In an embodiment, grooves 108 form an angle α of at least 10° from the longitudinal extent of internal surface 114. In an embodiment, grooves 108 form an angle α of at least 20° from the longitudinal extent of internal surface 114. In an embodiment, grooves 108 form an angle α of at least 30° from the longitudinal extent of internal surface 114. In an embodiment, grooves 108 form an angle α of at least 45° from the longitudinal extent of internal surface 114.

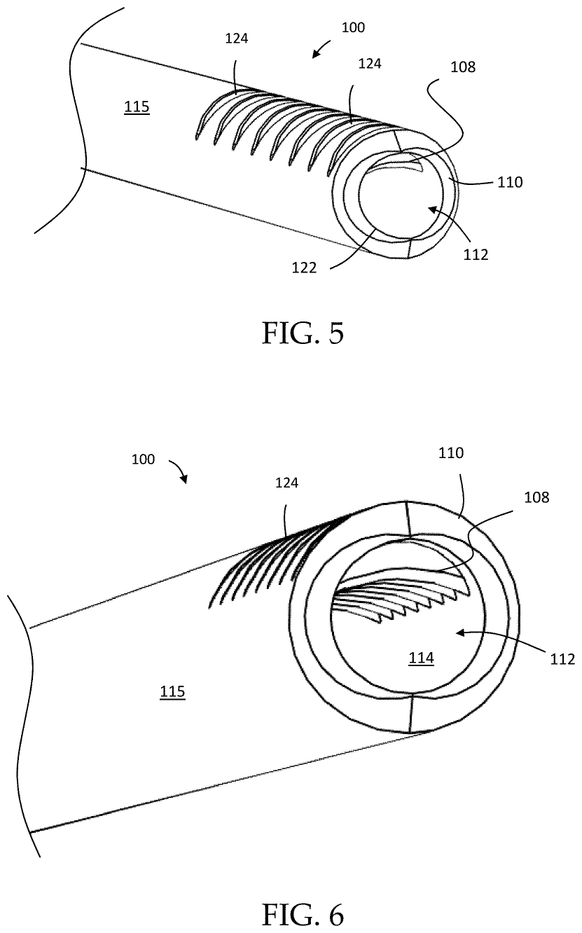

Referring now to FIGS. 5-11, an embodiment of biopsy needle 100 includes retaining members 108 created by crimping external surface 115. The crimping occurs in only a partial circumferential manner as illustrated by crimped sections 124. Crimped section 124 create the plurality of discontinuous, longitudinally spaced retaining members 108 extending inwardly towards central longitudinal axis 107 of elongated body 106. In other words, each retaining members 108 does not fully extend around a circumference of inner surface 114.

In an embodiment, retaining members 108 are angled with respect to longitudinal axis 107, when viewed from a top view, as depicted in FIG. 11. The referenced angle of retaining members 108 with respect to longitudinal axis 107 is identified by angle β (also referred to as an acute angle between the longitudinal axis and the retaining member). In an embodiment, angle β is roughly 75 degrees. In an embodiment, angle β is between 45 and 90 degrees. It should be noted that while FIG. 11 depicts an embodiment in which the retaining members 108 are created by crimping external surface 115, retaining members 108 may be angled with respect to longitudinal axis 107 regardless of how retaining members 108 are created or secured to the internal surface 114. The angled retaining members 108 move tissue in a distal direction when the biopsy needle is rotated to bring angled retaining members 108 into contact with the tissue sample residing within internal lumen 112.

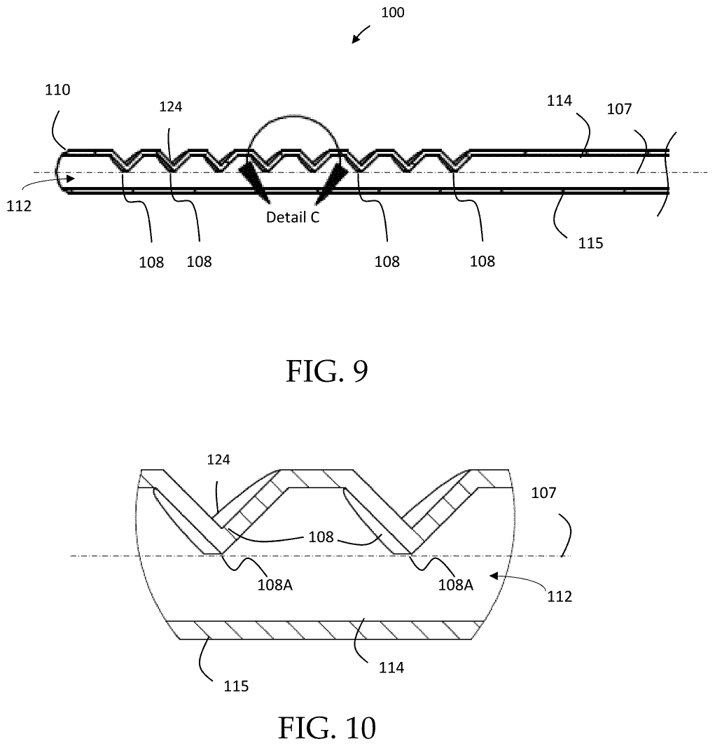

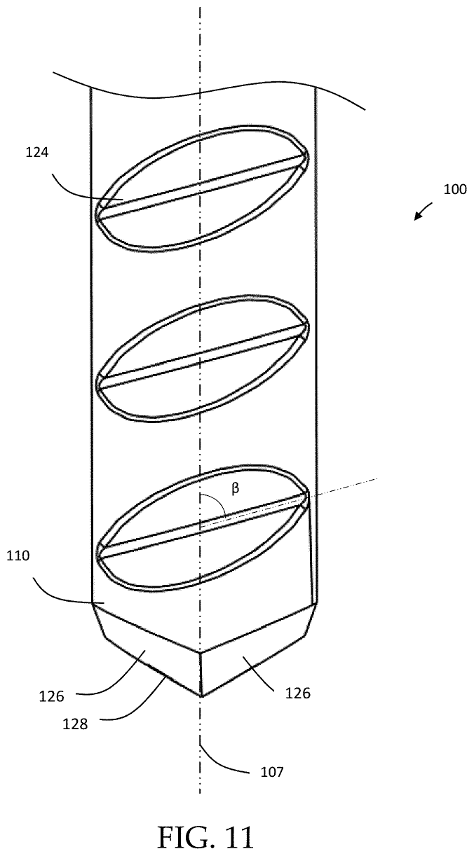

In an embodiment, as best depicted in FIGS. 9-10, retaining members 108 generally have a triangular shape when viewed from the cross-sectional profile views provided in FIGS. 9-10. A triangular shape helps to penetrate the sample tissue when retaining members 108 are rotated into engagement with the sample tissue.

Regardless of how the retaining members 108 are created, they have a pitch (i.e., the distance between successive corresponding points or lines between adjacent retaining members) of roughly 1-2 mm to ensure optimal engagement with tissue. In addition, each retaining member 108 occupies a cross-sectional area when in end view that is equivalent to 10-50% of an inner diameter 119 of internal lumen 112. In other words, distance 118 is equivalent to 50-90% of diameter 119. In an embodiment, distance 118 is equal to 20-25% of distance 119.

As best depicted in FIGS. 7-8 and 11, an embodiment includes cutting surfaces 126 located at distal end 110 of biopsy needle 100. Cutting surfaces 126 are beveled to create a sharp cutting edge 128 to aid the operator during insertion of the biopsy needle into a mass of tissue within a patient. As depicted in FIG. 7, an embodiment of cutting edges 128 have crescent profile shapes. These cutting edges occupy two opposing halves to create symmetry across a vertical longitudinal plane as shown in FIG. 8. This symmetry prevents unwanted rotation of the biopsy needle that could result from tissue contact with non-symmetric beveled cutting surfaces.

In an embodiment, the biopsy needle may also include a sample ejector or ejector funnel (not depicted). In an embodiment, the biopsy needle also includes a cannula and a trocar (not depicted) with optimum configurations, dimensions and orientations relative to each other to optimize penetration of the biopsy needle into bone or bone marrow with minimum patient trauma and enhanced reliability of obtaining a biopsy specimen.

Referring now to FIG. 12, the method of the present invention includes retrieving a biopsy needle (202); inserting the biopsy needle into tissue within a patient in a non-rotational manner (204); upon reaching an operator's preferred depth within the tissue, rotating the biopsy needle such that the plurality of retaining members are rotated into engagement with the portion of tissue within the internal lumen (206); and then removing the biopsy needle from the patient (208). In an embodiment, the degree of rotation is determined by the size and location of the retention flange 116 and/or the retaining members 108. The biopsy needle is rotated to orient the tissue within the shaft such that it resides at least partially behind retention flange 116 or to bring retaining members 108 into engagement with the tissue.

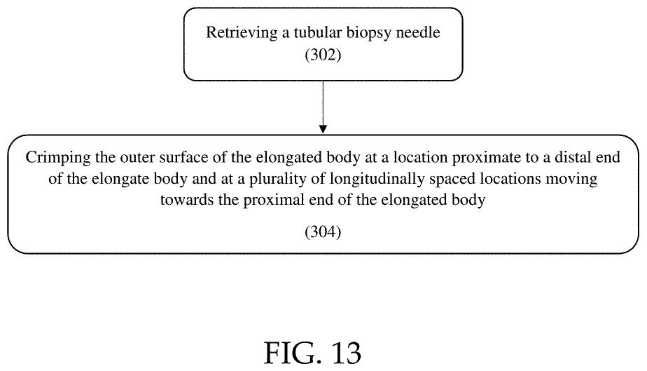

Referring now to FIG. 13, the present invention includes a method of manufacturing a biopsy needle having internal retaining members that overcomes the issues surrounding the inability to manufacture a relatively small biopsy needle having internal retaining members disposed within an even smaller internal lumen. An embodiment of the method of the manufacturing a biopsy needle having internal retaining members includes retrieving a tubular biopsy needle (302) and crimping the outer surface of the elongated body at a location proximate to a distal end of the elongate body and at a plurality of longitudinally spaced locations (304). In an embodiment, the retrieved biopsy needle is cylindrical with a generally smooth outer surface. In an embodiment, each crimping is longitudinally aligned with respect to the longitudinal axis of the biopsy needle.

The advantages set forth above, and those made apparent from the foregoing description, are efficiently attained. While the disclosure is susceptible to various modifications and implementation in alternative forms, specific embodiments have been shown by way of non-limiting example in the drawings and have been described in detail herein. Since certain changes may be made in the above construction without departing from the scope of the instant application, it is intended that all matters contained in the foregoing description or shown in the accompanying drawings shall be interpreted as illustrative and not in a limiting sense.

The disclosure is not intended to be limited to the particular forms disclosed. Rather, the disclosure is to cover all modifications, equivalents, and alternatives falling within the scope of the disclosure as defined by the following appended claims and their legal equivalents.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

It is also to be understood that the following claims are intended to cover all of the generic and specific features of the invention herein described, and all statements of the scope of the invention that, as a matter of language, might be said to fall therebetween.

What is claimed is:

1. A medical device configured to biopsy a tissue sample from a patient, comprising:
a biopsy needle having an elongated body with an outer surface and an internal lumen established by an inner surface;
the inner surface including a plurality of discontinuous, longitudinally spaced retaining members extending radially towards a central longitudinal axis of the elongated body, wherein each of the retaining members does not fully extend around a circumference of the inner surface;
a distal aperture in a distal end of the biopsy needle providing access to the internal lumen;
whereby the biopsy needle can be inserted into tissue in the patient, such that a portion of tissue passes through the distal aperture and into the internal lumen, and then the biopsy needle can be rotated to cause the plurality of retaining members to engage and retain the portion of tissue within the internal lumen when the biopsy needle is retracted out of the patient.

2. The medical device of claim 1, further including a handle integrated with or attachable to a proximal end of the biopsy needle to enable an operator to rotate the biopsy needle.

3. The medical device of claim 1, further including an introducer needle configured to house the biopsy needle within an internal lumen when the medical device is inserted into the patient.

4. The medical device of claim 3, wherein the biopsy needle can distally extend a predetermined distance from a distal end of the introducer needle and the biopsy needle includes the plurality of retaining members longitudinally spaced along a length of the biopsy needle that is equivalent to the predetermined distance that the biopsy needle can distally extend from the distal end of the introducer needle.

5. The medical device of claim 1, wherein each of the plurality of retaining members is angled with respect to the central longitudinal axis, such that an acute angle between the longitudinal axis and each retaining member is between 45 and 90 degrees.

6. The medical device of claim 1, wherein the distal end of the biopsy needle includes a beveled cutting surface.

7. A method to biopsy a tissue sample from a patient, comprising:
retrieving a biopsy needle, wherein the biopsy needle includes:
an elongated body with an outer surface and an internal lumen established by an inner surface;
the inner surface including a plurality of discontinuous, longitudinally spaced retaining members extending radially towards a central longitudinal axis of the elongated body, wherein each of the retaining members does not fully extend around a circumference of the inner surface;
a distal aperture in a distal end of the biopsy needle providing access to the internal lumen;
inserting the biopsy needle into tissue within a patient in a non-rotational manner, such that a portion of tissue passes through the distal aperture and into the internal lumen;
upon the biopsy needle reaching an operator's preferred depth within the tissue, rotating the biopsy needle such that the plurality of retaining members are rotated into engagement with the portion of tissue within the internal lumen;
retracting the biopsy needle from the patient;
whereby the plurality of retaining members retain the portion of tissue within the internal lumen during retraction.

8. The method of claim 7, wherein rotating the biopsy needle includes at least a 90-degree rotation about the central longitudinal axis.

9. The method of claim 7, wherein a proximal end of the biopsy needle is integrated with or attachable to a graspable handle to enable an operator to rotate the biopsy needle.

10. The method of claim 7, wherein each of the plurality of retaining members is angled with respect to the central longitudinal axis, such that an acute angle between the longitudinal axis and each retaining member is between 45 and 90 degrees.

11. The method of claim 7, wherein the distal end of the biopsy needle includes a beveled cutting surface.

12. A method of manufacturing a biopsy needle, comprising:
retrieving a tubular biopsy needle having an elongated body with an outer surface and an internal lumen established by an inner surface; and
crimping the outer surface of the elongated body at a location proximate to a distal end of the elongate body and at a plurality of longitudinally spaced locations, wherein each crimping creates a retaining member that extends within the internal lumen towards a central longitudinal axis of the elongated body and each retaining member does not fully extend around a circumference of the inner surface.

13. The method of claim 12, wherein each crimping occurs at an angle with respect to the central longitudinal axis, such that an acute angle between the longitudinal axis and each retaining member is between 45 and 90 degrees.

14. The method of claim 12, wherein each crimping occurs is longitudinally aligned along the outer surface of the elongated body.

15. The method of claim 12, wherein each crimping creates a retaining member extending generally the same distance towards the central longitudinal axis of the elongate body.
